(19) United States
(12) Patent Application Publication
(10) Pub. No.: US 2009/0281397 A1
(43) Pub. Date: Nov. 12, 2009

(54) Device and method for measuring vaginal and perivaginal physiological signals, particularly blood flow and the perivaginal muscles

(76) Inventor: Pierre LAVOISIER, Rasteau (FR)

Correspondence Address: YOUNG & THOMPSON, 209 Madison Street, Suite 500, ALEXANDRIA, VA, 22314, US

(21) Appl. No.: 12/435,575

(22) Filed: May 5, 2009

Related U.S. Application Data
(60) Provisional application No. 61/050,320, filed on May 5, 2008.

(30) Foreign Application Priority Data
May 5, 2008 (FR) 08/02471

(51) Int. Cl. A61B 5/00; A61B 5/103

(52) U.S. Cl. 600/301; 600/591

(57) ABSTRACT

The measuring device according to the present invention includes a cylindrical probe (2) including a cylindrical longitudinal body (4) equipped with a flange (5) forming a stop, a pressure sensor (8) placed according to a direction longitudinal to the surface of the cylindrical body (4), an electronic card (15) disposed inside the cylindrical body (4) to which the pressure sensor (8) is connected, and communication means (16, 17) longitudinally traversing the probe (2) and allowing the inner part of the vagina to be connected to the outer in order to balance the intravaginal and extravaginal pressures.

The present invention relates to a measuring method enabling pressures and variations in pressure to be measured during perivaginal muscle contraction, enabling vaginal and clitoral circulation to be measured and the progression in these types of circulation to be controlled, as well as a device for implementing the method.

Measurements taken by the device are performed either while the patient sleeps or during the day and allow, according to the results obtained, the cause of dysfunctions to be determined and their progression to be controlled.

Measuring perivaginal muscle tone during sleep allows organic sexual dysfunctions to be dissociated from psychogenic sexual dysfunctions. Patients with an organic sexual dysfunction do not have variations in muscle tone or blood flow during paradoxical phases of sleep.

Also, the measuring device which is the object of the invention enables the rehabilitation of patients to be controlled in the case of urinary stress incontinence as well as rehabilitation in the case of anorgasmy.

During perineal rehabilitation in the woman, abdominal contractions interfere with the measurement of pressure caused by the perivaginal muscles.

In fact, to improve the amplitude of the signal seen on the screen, patients tend to contract not only their perivaginal muscles, which is the goal, but also their abdominal muscles, which artificially increases the result and completely disrupts the measurements.

This abdominal contraction increases the intravaginal pressure, which increases the pressure on the measuring device, especially if it is an inflatable probe that hermetically closes the vaginal opening.

The object of the measuring method and the device for its implementation is to remedy the disadvantages stated above.

The measuring method allowing vaginal and perivaginal physiological signals, such as pressures and pressure variations during contraction of the perivaginal (PV) muscles, but also perivaginal pulse, clitoral pulse, clitoral intracavernous pressure, vaginal temperature and any other relevant physiological signal to be measured, consists of:
Placing a sensor comprising means for receiving the pressure and pressure variations during a contraction of the perivaginal (PV) muscles, and at least one transducer allowing the pressure signal issued from the pressure receiving means to be transformed into an electrical signal,
And connecting the sensor to an electronic card or a housing comprising means for amplifying and recording the signal from the transducer, and means for analyzing the signal by a microcontroller allowing the state of perivaginal (PV) muscle fatigue to be measured and the variations in muscle strength of the perivaginal (PV) muscles to be measured.

The measuring method consists of measuring variations in intracavernous pressure (ICP) in order to quantify the variations in perivaginal tone and in arterial and venous circulation both day and night.

The measuring method consists of measuring the intracavernous pressure (ICP) when the pressure progressively reaches mean systolic pressure.

The measuring method consists of measuring the increase in arterial inflow in the clitoral corpus cavernosum and the reduction in venous outflow during the vascular phase, which is the first clitoral erection phase.

The measuring method consists of measuring variations in the contraction and rigidity of the perivaginal (PV) and erector (EM) muscles surrounding the clitoral corpus cavernosum during the muscular phase, which is the second clitoral erection phase.

The measuring method consists of continuously measuring the arterial inflow and the venous outflow by measuring the pulse pressure (PP) and the pulse volume (PV).

The measuring method consists of measuring the Young's modulus of the clitoral corpus cavernosum allowing the compliance of said clitoral corpus cavernosum to be evaluated.

The measuring method consists of measuring the leak from the intracavernous pressure (ICP) curve.

The measuring method consists of measuring the maximum contraction (Pmax), the pressure gradient (Delta P and mean delta), the surface under the curve of each contraction, the number of peaks and the width of each peak.

The measuring method consists of measuring perivaginal (PV) muscle fatigue by measuring the angle (a).

The measuring method consists of measuring the peaks of the perivaginal (PV) muscle contractions and the variations in pulse pressure.

The method for treating a patient with sexual dysfunctions, said patient having been diagnosed thanks to perivaginal and clitoral measurements taken both day and night, said measurements consisting of measurements of perivaginal (PV) muscle strength and of arterial and venous variations, consists of:

Placing a sensor comprising means for receiving the pressure and pressure variations during a contraction of the perivaginal (PV) muscles, means for electro stimulating the perivaginal (PV) muscles and at least one transducer allowing the pressure signal issued from the pressure receiving means to be transformed into an electrical signal,
And connecting the sensor to an electronic card or to a housing comprising means for amplifying and recording the signal from the transducer, means for analyzing the signal by a microcontroller allowing the electro stimulation to be controlled as a function of the state of perivaginal (PV) muscle fatigue and the intensity and muscular rehabilitation to be controlled through sound, visual or tactile patterns indicating to the patient the intensity and duration of the muscle contraction to be produced and the duration of the rest period between two contractions.

The method for treating a patient with sexual dysfunctions consists in that the electro stimulation means are constituted of electrodes controlled by the patient in order to vary the intensity.

The method for treating a patient with sexual dysfunctions consists in that the diagnosis of organic dysfunction is established during the night by the measuring device.

The method for treating a patient with sexual dysfunctions consists in that the rehabilitation is specially indicated for patients presenting a muscular pathology having a normal vascular phase, but also for those having a mixed vascular pathology of arterial or venous and muscular origin.

The method for treating a patient with sexual dysfunctions consists in that the patient is placed on an examination bed or on her bed, the probe is introduced into her vagina such that the electro stimulation plates and the pressure sensor are against the perivaginal (PV) muscles, which is achieved thanks to the flange that presses on the vulva, allowing it to be positioned properly.

The method for treating a patient with sexual dysfunctions consists in that the patient is placed on an examination bed or on her bed, the open ring is introduced into her vagina such that the branches equipped with electro stimulation electrodes are against the perivaginal (PV) muscles.

The method for treating a patient with sexual dysfunctions consists in that the electro stimulation system of the electronic card or the housing is started, and the patient adjusts the intensity of the stimulation either by using a potentiometer placed on the housing or by using a virtual button situated on the screen of either the computer, a PDA, or a mobile telephone.

The method for treating a patient with sexual dysfunctions consists in that the electro stimulation is continued until the end of the session, as with any muscular rehabilitation, since it improves the performance of the perivaginal (PV) muscle rehabilitation as well as the perivaginal vasodilation.

The method for treating a patient with sexual dysfunctions consists in that the patient verifies that the voluntary contractions cause an elevation in pressure on pressure sensor which proves that the right muscles are contracted, since only the perivaginal (PV) muscles should be contracted, without contraction of the abdominal muscles, which disturbs the measurements.

The method for treating a patient with sexual dysfunctions consists in that the patient then sees a mask of muscle contractions on the screen that she is invited to follow, indicating the optimal duration and height of the contraction that must be made.

The method for treating a patient with sexual dysfunctions consists in that the muscle contraction mask is adjustable and established from the fatigue curve of the perivaginal (PV) muscles.

The method for treating a patient with sexual dysfunctions consists in that modifications in the mask indicating the modalities of the contraction to be performed will be calculated either in real time by the microcontroller during the session or between two sessions when this analysis will be done by the analysis and telecontrol center (ATC).

A method for treating a patient with problems with urinary stress incontinence consists in that the perineal rehabilitation is obtained by means of the sensor with electro stimulation of the vagina through electro stimulation means positioned on the sensor, electro stimulation of the vagina allowing the perivaginal (PV) muscles surrounding the vagina to be strengthened and the arterial circulation to be increased.

A measuring device for implementing the method consists in that the sensor is constituted of a probe comprising a cylindrical longitudinal body equipped with a flange forming a stop, a pressure sensor placed according to a direction longitudinal to the surface of the cylindrical body, an electronic card disposed inside the cylindrical body and to which the pressure sensor is connected, and communication means longitudinally traversing the probe that are constituted of at least one longitudinal channel provided on the periphery of the cylindrical body and traversing the flange to enable the inner part of the vagina to be connected to the outer in order to balance the intravaginal and extravaginal pressures.

The measuring device for implementing the method comprises a sensor constituted of a probe comprising a cylindrical longitudinal body equipped with a flange forming a stop, a pressure sensor placed according to a direction longitudinal to the surface of the cylindrical body, an electronic card disposed inside the cylindrical body and to which the pressure sensor is connected, and communication means longitudinally traversing the probe that are constituted of a channel provided inside the cylindrical body and longitudinally traversing the latter and the flange to enable the inner part of the vagina to be connected to the outer in order to balance the intravaginal and extravaginal pressures.

The measuring device for implementing the method comprises a sensor constituted of a probe comprising a longitudinal cylindrical body equipped with a flange forming a stop, a pressure sensor placed according to a direction longitudinal to the surface of the cylindrical body, an electronic card disposed inside the cylindrical body and to which the pressure sensor is connected, and communication means longitudinally traversing the probe that are constituted of, first, at least one longitudinal channel provided on the periphery of the cylindrical body and traversing the flange and, second, by a channel provided inside the cylindrical body and longitudinally traversing the latter and the flange, said communication means allowing the inner part of the vagina to be connected to the outer in order to balance the intravaginal and extravaginal pressures.

A measuring device for implementing the method comprises a probe comprising, at the surface of the cylindrical body, electro stimulation plates connected to the electronic card.

The measuring device comprises electro stimulation plates positioned parallel to the longitudinal axis of said cylindrical body.

The measuring device comprises electro stimulation plates presenting a circular or spherical profile.

The measuring device comprises a pressure sensor constituted of a metallic plate that is fixed in a groove provided in the cylindrical body, said groove being filled with a composite elastic material allowing the movements or elastic deformations of this plate to be absorbed, while the inner face of the metallic plate is integral with a strain gauge or transducer allowing the pressure signal issued from the metallic plate to be transformed into an electrical signal.

The measuring device comprises a pressure sensor constituted of a piezoelectric film or transducer fixed at the level of the groove filled with a composite elastic material.

The measuring device comprises a probe comprising, inside the cylindrical body, a cutout permitting an electronic card to be placed and held.

The measuring device comprises a probe comprising all the sensors necessary for the vaginal physiological measurements connected with the electronic card.

The measuring device comprises an electronic card that may be connected to a housing containing an amplifier, an analog to digital converter and a microprocessor.

The measuring device for implementing the measuring method comprises a sensor constituted of:

An open ring comprising two branches in an arc of circumference interconnected by a flexible element,
And a strain gauge integral with the flexible element and connected to a housing or to an electronic card.

The measuring device comprises a strain gauge disposed on the inner face of the flexible element such that the latter is found inside the open ring to prevent any risk of injuring the vagina of the patient.

The measuring device comprises flat, plate-shaped branches made in a material with high elastic deformation strength, or a material that is said to be not very deformable.

The measuring device comprises a flexible element connecting the branches that is constituted of a thin plate of metal presenting significant elastic deformation characteristics.

The measuring device comprises an open ring comprising, on the outer face of branches, electro stimulation means that are constituted of electrodes connected to a housing containing an amplifier, an analog to digital converter, an electronic card, a microprocessor and a microcontroller managing the electro stimulation.

The attached drawings, given by way of example, allow the invention, the characteristics that the invention presents and the advantages that the invention is likely to bring to be better understood:

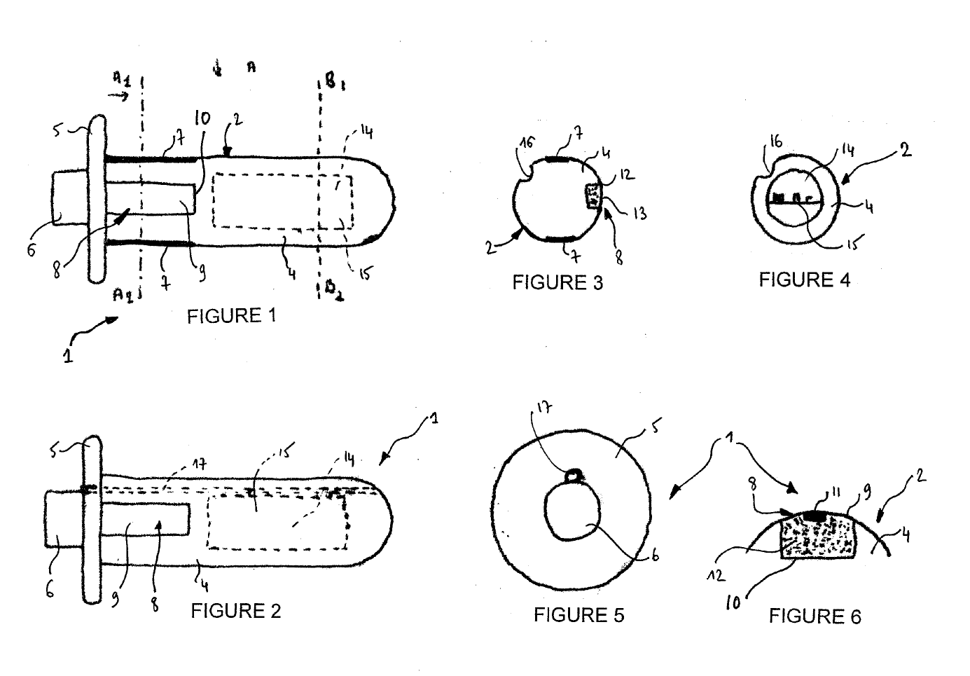

FIGS. 1 and 2 are side views illustrating the measuring device according to the present invention.

FIGS. 3 to 6 are cross-sectional schematic views representing the measuring device according to the present invention.

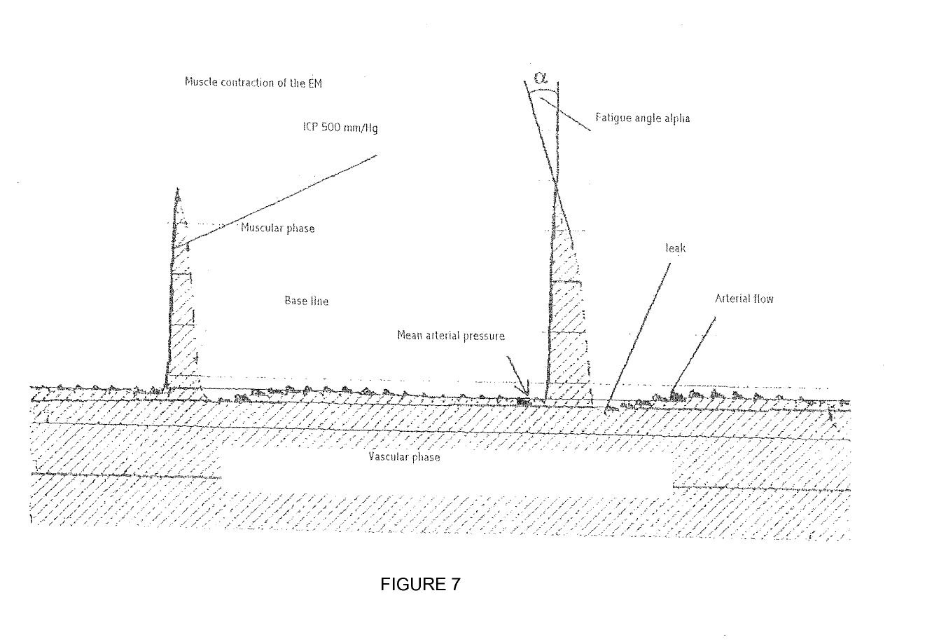

FIG. 7 is a diagram showing the variation in pressure measured by the measuring and control device according to the present invention.

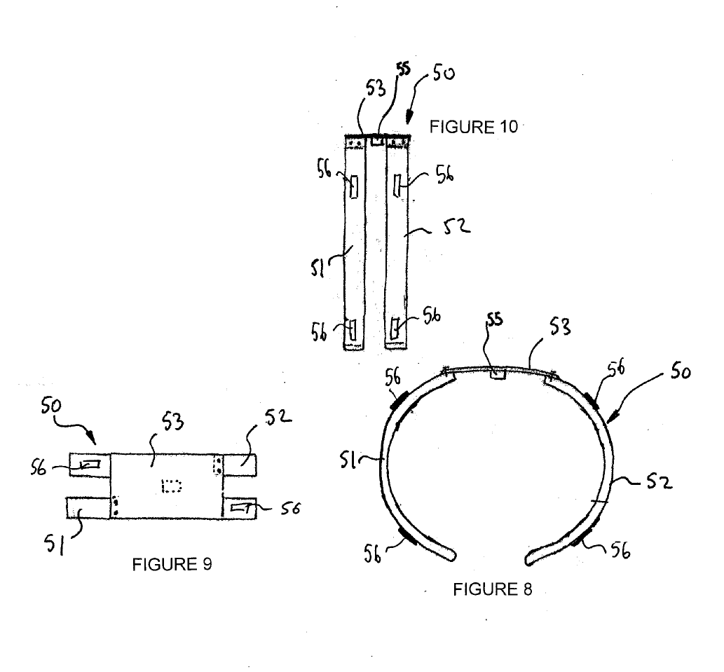

FIGS. 8 to 10 are views illustrating a measuring device comprising a sensor made from an open ring according to the present invention.

FIGS. 1 to 6 show a measuring device 1 allowing pressures and pressure variations during perivaginal (PV) muscle contractions to be measured.

The measuring device 1 comprises a sensor that is comprised of a probe 2 comprising means to monitor the pressure and variations in pressure of the muscular strength of the perivaginal (PV) muscles.

Probe 2 is comprised of a rigid cylindrical body 4 made from a non-deformable plastic material.

The cylindrical longitudinal body 4 is equipped with a flange 5 forming a stop and allowing said probe to be positioned inside the vagina in a given position and a flat bar 6 ensuring that said probe is grasped and held.

Flange 5 is positioned between the cylindrical longitudinal body 4 and the flat bar 6. The latter may be provided with an outer dimension that is identical to or less than that of the cylindrical longitudinal body 4.

Probe 2 comprises either two or four electro stimulation plates 7 near the flange 5 and fixed on the cylindrical body 4. The latter are positioned at the surface of the cylindrical body 4 and according to a direction that is parallel to the longitudinal axis of said cylindrical body 4.

The electro stimulation plates 7 are made of a conductive material that may be either a conductive alloy or elastomer. The electro stimulation plates 7 may present a circular or spherical profile or any other shape allowing good electro stimulation and good pressure measurement. In fact, the electro stimulation plates 7 may act as pressure sensors.

Probe 2 comprises means to monitor the pressure and pressure variations during perivaginal (PV) muscle contractions that are constituted of a pressure sensor 8 placed according to a direction longitudinal to the surface of the cylindrical body 4 and between the electro stimulation plates 7.

The pressure sensor 8 is constituted of a metallic plate 9 that is fixed in a groove 10 provided, from the flange 5, in the cylindrical body 4 of the probe 2.

The metallic plate 9 is integral, on its inner face, with a strain gauge 11 or transducer allowing the pressure signal issued from the metallic plate 9 to be transformed into an electrical signal.

Prior to fixation of the metallic plate 9, the groove 10 is filled with a composite elastic material 12 allowing the movements or elastic deformations of said plate 9 to be absorbed (FIGS. 1, 2, 6).

The pressure sensor 8 may be comprised of a piezoelectric film 13 or transducer fixed at the level of the groove 10 that is filled with the composite elastic material 12. In this case, the piezoelectric film 13 does not comprise a strain gauge, since the latter, by its constitution, allows the pressure signal issued from said film to be transformed into an electrical signal (FIG. 3).

Inside the cylindrical body 4, probe 2 comprises a cutout 14 permitting an electronic card 15 to be placed and held, to which the electro stimulation plates 7 and the pressure sensor 8 or any other sensor or other electrical or mechanical stimulators are connected (FIGS. 1, 2 and 4).

The electronic card 15 comprises means for recording and amplifying the signal from the strain gauge 11 or piezoelectric film 13, each forming a transducer.

The electronic card 15 comprises means for analyzing the signal by a microcontroller allowing the electro stimulation of plates 7 to be controlled according to the perivaginal (PV) muscle fatigue state and the muscular strength intensity to be controlled through sound, visual or tactile patterns indicating to the patient the muscular contraction intensity and duration to be produced and the duration of the rest period between two contractions.

The electronic card 15 comprises at least one battery that is possibly rechargeable by induction ensuring the necessary autonomy of the probe 2 and its associated electronics.

On the periphery of the cylindrical body 4, probe 2 comprises at least one longitudinal channel 16 traversing flange 5 by a conduit constituting pressure communication means between the inner and outer parts of the vagina in order to balance the intravaginal and extravaginal pressures (FIGS. 3 and 4).

These communication means may also be made through at least one channel 17 provided inside the cylindrical body 4 and first leading to the outside and above the flat gripping bar 6 and second inside the vagina at the level of the free end of said cylindrical body (FIGS. 2 and 5).

Also, at the surface of the cylindrical body 4, probe 2 may comprise sensors connected to the electronic card 15 measuring physiological signals, that is, pressure, temperature, lubrication and blood flow, particularly by photoplethysmography, as well as all relevant physiological signals.

When probe 2 comprises two electro stimulation plates 7, the latter may be used at the same time for electrical stimulation and for measuring pressure, the microcontroller managing a stimulation time and then a measuring time.

When probe 2 is introduced inside the vagina, it is observed that the peripheral flange 5 of the probe 2 presses against the vulva of the latter, guaranteeing that the electro stimulation plates 7 and the pressure sensor 8 are always in contact with the same part of the vaginal wall and more particularly are against the perivaginal (PV) musculature of the patient.

The electro stimulation plates 7 are connected to the electronic card 15 comprising an electro stimulation system allowing weak electrical pulses to be sent to said electro stimulation plates 7 in order to ensure controlled electro stimulation of the perivaginal (PV) muscles, while data received by the electronic card 15 are sent to a housing.

It is noted that the electrical stimulation of the electro stimulation plates 7 is activated by the patient so as to be perceived without ever being painful. The patient may vary the intensity at any time.

FIGS. 8 to 10 illustrate a measuring device 1 that is constituted of a sensor formed by an open ring 50 comprising two branches 51, 52 in an arc of circumference interconnected at the top of the circular profile by a flexible element 53.

Branches 51, 52 are plate-shaped flat branches made in a material with high elastic deformation strength, or a material that is said to be not very deformable.

The flexible element 53 connecting the branches 51, 52 is constituted of a thin plate of metal presenting significant elastic deformation characteristics. On one of its faces, the flexible element 53 comprises a strain gauge 55 that is connected by any means either to an electronic card or to a housing, not shown, of the measuring device 1.

The strain gauge 55 is disposed on the inner face of the flexible element 53 so that the latter is found inside the open ring 50 to prevent any risk of injury to the vagina of the patient.

The housing contains an amplifier, an analog to digital converter, a microprocessor and a removable memory card.

Branches 51, 52 of the open ring 50 each comprise a free end on which pressure may be exerted to obtain a slight deformation of the flexible element 53 so as to be able to place ring 50 inside the vagina of the patient.

The structure and composition of the open ring 50 enables it to be adapted to different volumes and circumferences of the vagina without restricting the blood circulation within the latter.

The open ring 50 also comprises electro stimulation means that are constituted of electrodes 56 positioned on the outer face of branches 51, 52.

The electrodes 56 are made of a conductive material that may be either a conductive alloy or elastomer. The electrodes 56 may present a circular or spherical profile or any other shape allowing good electro stimulation and good pressure measurement. In fact, the electrodes 56 may act as pressure sensors.

Electrodes 56 are connected to the outer housing, not shown, comprising an electro stimulation system allowing weak electrical pulses to be sent to said electrodes 56 in order to ensure controlled electro stimulation of the perivaginal (PV) muscles, while the data received are sent to said housing.

### Perivaginal (PV) Muscles, Clitoral Erection and Physiological Measurements

Atrophy of the perivaginal (PV) muscles comprising the erector muscle (EM) promotes female anorgasmy. Urinary stress incontinence is also connected to atrophy of the perivaginal musculature and the associated urethral sphincters.

During sexual excitation (SE), the intracavernous pressure (ICP) progressively reaches the mean systolic pressure, or 100 to 120 mm/hg. The maximum volume of the clitoral corpus cavernosum is reached when the elasticity is blocked by the tunica albuginea.

The tunica albuginea is a membrane with low elasticity that surrounds the clitoral corpus cavernosum, preventing its dilation, which enables the pressure to be elevated.

The vascular phase is the first phase of clitoral erection, or tumescence, a consequence of the increase in inflow (arterial) into the clitoral corpus cavernosum associated with a reduction in outflow (venous).

The second phase, known as the muscular phase, becomes effective once the first phase is obtained. Contraction of the perivaginal (PV) muscles surrounding the clitoral corpus cavernosum causes the intracavernous pressure (ICP) to rise, which constitutes the clitoral erection. This elevation in intracavernous pressure (ICP) is only possible thanks to the tunica albuginea preventing the dilation of the clitoral corpus cavernosum.

To obtain a clitoral erection, thus having a high intracavernous pressure (ICP) due to contraction of the perivaginal (PV) muscles, the vascular phase must precede the muscular phase.

In fact, if the vascular phase is not achieved, the tension exerted on the tunica albuginea is low and consequently its elasticity is high. The muscle contraction is then weak or not effective.

If the intracavernous pressure (ICP) is low, the Young's modulus of the tunica albuginea is low, leading to a slight variation in intracavernous pressure (ICP), (delta ICP), caused by the contraction of the perivaginal (PV) muscles, since the pressure variation (delta ICP) is absorbed by the elasticity of the tissues.

On the other hand, at the end of the vascular phase, if the tumescence is complete, the tension exerted on the tunica albuginea is high, its elasticity is low, the Young's modulus is high, causing a pressure variation (delta ICP) that is poorly absorbed by the elasticity of the tissues and thus significant variations in intracavernous pressure (ICP).

The three most frequent causes of female sexual dysfunction are:
Arterial causes due to reduction in inflow,
The causes of leaks due to the absence of reduction in outflow.
Muscular causes due to a reduction in muscular strength of the perivaginal (PV) muscles by a lesion in the perivaginal tissues, particularly during childbirth.

### Arterial Causes or Inflow Measurement

Measuring inflow is conventionally done by the Doppler effect. The Doppler effect measures the speed of blood but does not measure the flow, unless the diameter of the vessels is known, which is possible but not very precise. Variations in flow can be measured by measuring the surface under the Doppler signal curve, but these are only variations.

In addition, Doppler effect measurements are not adapted to continuous measurements since the investigator is required to be continuously present. This is due to the fact that the position of the Doppler probe must be maintained with a precise angle with relation to the artery, the least movement “losing” the signal.

Doppler is thus poorly adapted to continuous measurements, especially during sleep.

This is the reason for which the measuring device 1 according to the present invention ensures continuous measurements, particularly during sleep, of inflow by measuring the pulse volume (PV) by photoplethysmography (PVP), but also by measuring variations in pulse pressure (PP) by tonometry.

The measuring device 1 allows the cavernosal artery flow or inflow to be measured. This method is based on measuring the pulse pressure (PP). In fact, during nocturnal or diurnal recordings, we observe, after absorption of a vasodilator, that variations in pressure generated by the pulse are visible. These pressure variations correlate with the blood flow under certain conditions.

Measuring device 1 allows the inflow to be continuously measured according to two different methods:

Either by pulse volume (PV), which requires two different sensors, a pressure sensor to measure the intracavernous pressure (ICP) and intravaginal pressure and the other by photoplethysmography (PPL) to measure the flow, the PPL being either in the probe or transported to the outside.
Or by pulse pressure (PP) with a single sensor that simultaneously measures the intracavernous pressure (ICP) and intravaginal pressure and variations in the pulse pressure (PP).

### The Causes of Leaks Due to the Absence of Reduced Outflow.

Venous leaks, or cavernosal leaks, or cavernosal venous leaks are the main causes of [the absence of] reduced outflow.

This is due to a reduction in elasticity or compliance of the clitoral corpus cavernosum that restricts its dilation (and thus clitoral erection), but especially hinders the outflow reduction.

In fact, the venous network of the clitoral corpus cavernosum, which enables the outflow, is at the periphery of the clitoral corpus cavernosum under the tunica albuginea, and is thus situated between the clitoral corpus cavernosum and the tunica albuginea.

During dilation of the clitoral corpus cavernosum, whose compliance is high, the venous network is compressed between the clitoral corpus cavernosum and the tunica albuginea, whose compliance is low.

If, for pathological reasons, the compliance of the clitoral corpus cavernosum diminishes, venous compression cannot take place. The outflow is not reduced, thus we speak of a venous leak that is in fact a cavernosal venous leak not connected to a venous pathology but rather to a cavernosal pathology.

In this case, even if the outflow is sufficient, the intracavernous pressure (ICP) cannot rise if the leak is significant. In case of a slight leak, the inflow may compensate for this loss by increasing the flow.

The measuring device 1 allows the leak from the curve of the intracavernous pressure (ICP) to be measured.

The curve illustrated in FIG. 7 represents the intracavernous pressure (ICP), wherein the base line indicates the level of the intracavernous pressure (ICP) outside of a contraction of the perivaginal (PV) muscles.

As the sudden elevation in pressure corresponds to a contraction of the perivaginal (PV) muscles (muscle contraction), it perfectly correlates with the electromyographic activity of the perivaginal (PV) muscles.

During sleep, these contractions are involuntary and during a rehabilitation session they are voluntary, but the appearance of the curve is identical.

The measuring device 1 enables a drop in intracavernous pressure (ICP) below the base line just after a voluntary contraction (muscle contraction), then an increase in several seconds to regain the level of pressure obtained just before the contraction, to be demonstrated.

In fact, when the intracavernous pressure (ICP) is stable, the value of the intracavernous pressure (ICP) is none other than the mean systolic pressure; inflow is then equal to outflow since the intracavernous pressure (ICP) is stable.

The sudden drop in intracavernous pressure (ICP) is caused by the muscle contraction. In fact, outside of these contractions, a sudden drop in the intracavernous pressure (ICP) is never observed. This pressure drop is due to a slight reduction in the volume of blood contained in the clitoral corpus cavernosum.

Thus, this is a leak caused by a muscle contraction that forces out a quantifiable volume of blood. In fact, we can count the number of heart beats necessary for its filling, knowing the pulse volume we can deduce the leak volume during a contraction.

For a normal patient, the clitoral corpus cavernosum compliance is high and the intracavernous pressure (ICP) sufficiently compresses the venous network to allow the outflow to remain equal to the inflow, this is the physiological flow.

During a contraction of the perivaginal (PV) muscles, the sudden elevation in intracavernous pressure (ICP) is due to an extrinsic compression of the tunica albuginea by the perivaginal (PV) muscles. This pressure is transmitted to the inner face of the tunica albuginea and then to the clitoral corpus cavernosum, which causes a sudden elevation in the intracavernous pressure (ICP) and a strong compression of the venous network.

On the other hand, if the compliance is low, the transmission of pressures is not sufficient to obtain a complete compression of the veins, which forces a volume of blood outside of the clitoral corpus cavernosum.

The leak is measured from the surface of the triangle observed just after a voluntary muscle contraction.

For a pathological patient, especially if a leak is suspected, measuring the Young's modulus of the clitoral corpus cavernosum becomes fundamental since it enables the clitoral corpus cavernosum compliance to be evaluated, and thus the cause of the leak to be confirmed.

Several techniques exist to measure the Young's modulus of the clitoral corpus cavernosum. Of course this measurement, as well as the evaluation of the clitoral corpus cavernosum compliance, may be carried out with the measuring device 1 according to the present invention.

Muscular causes due to a reduction in muscular strength of the perivaginal (PV) muscles

Muscle contraction is visible on the curve illustrated in FIG. 7; this is a muscle contraction that perfectly correlates with the electromyographic activity of the perivaginal (PV) muscles.

There is even a causal link between the peaks recorded and the contraction of the perivaginal (PV) muscles. The contraction of the perivaginal (PV) muscles participates in clitoral erection.

The measuring device 1 according to the present invention enables the following measurements to be taken, either at night or during an artificial clitoral erection:
Pmax: Maximum contraction.
delta P: The pressure gradient recorded above the base line.
The mean delta during a day session or a night.
The surface under the curve of each contraction objectifying the work provided by the muscle and the average of these surfaces.
The number of peaks.
The width of each peak measured at its base and their average.

### Nocturnal Recording Procedure

The measuring device 1 according to the present invention allows the rehabilitation of patients with orgasmic dysfunction or urinary incontinence to be controlled.

Measuring variations in the perivaginal tone of the perivaginal arterial flow of nocturnal clitoral erections is essential for determining the sexual dysfunction type, that is, psychogenic sexual dysfunctions versus organic sexual dysfunctions.

The measuring device 1 utilized for nocturnal measurements is practically the same as that utilized for daytime perineal rehabilitation, it differs in that it is not connected to a computer screen, data are stored in a removable memory and there is no electro stimulation system.

The analysis software is also a little different, since it notably calculates the duration of the elevations in perivaginal tone of the perivaginal arterial flow and the clitoral erections, their numbers are established and the zooms done on each erection are calculated.

The recording is done at home, without sleep recording, but may also take place in a sleep laboratory.

If the recording is done at home, the housing is entrusted to the patient, who comes to pick it up at the physician's office.

Prior to this, the physician will have programmed the housing in order to record the characteristics of the patient (date, last name, first name, age, tobacco use, her pathologies are detailed, glycemia, cholesterol, medications, and all other information likely to intervene in her sexual function).

Also, the physician will be able to isolate, by using the analysis and telecontrol center (ATC), groups of patients having the same type of pathology known as muscular, vascular, mixed or normal “Clusters,” for which means and standard deviations will be established, each new recording will be automatically added to its cluster, and its statistics will be updated.

The physician, after having recorded the data of the patient, verifies the battery charge, calibration and proper functioning of the recording. He disconnects the housing from his computer and entrusts it to the patient.

At bedtime, the patient places probe 2 into her vagina, which is connected by a wireless or wire connection to the housing, and a red LED blinks, indicating that the recording has started. An automatic zero reset of the pressure sensor 8 is performed by the software after several minutes of recording.

After waking up, the patient verifies that the LED is still lit, attesting to the fact that the recording was not interrupted during the night. The patient turns the housing off.

The patient starts the same operations described above over again on the following nights.

After three consecutive nights, the patient brings the housing back to the physician, who transfers the data contained in the housing memory to his computer.

The data are partially analyzed by the software and then sent, via the Internet, in the database of the analysis and telecontrol center (ATC).

A hard copy of the curves, zooms and calculations of each night will be printed, and the complete analysis will be sent by the analysis and telecontrol center (ATC).

### Sexual Dysfunction Treatment Procedure

Measuring device 1 according to the present invention enables patients with sexual dysfunction and incontinence to be treated. This treatment may be carried out by rehabilitation either at the attending physician's office or at the patient's home.

When the analysis of the organic sexual dysfunction is established by the measuring device 1, the latter also enables its rehabilitation.

Rehabilitation is especially indicated for patients presenting a muscular pathology having a normal vascular phase, but also for those having a mixed vascular pathology of arterial and muscular origin.

The patient is placed on an examination bed or on her bed, probe 2 is introduced into her vagina such that the electro stimulation plates 7 and the pressure sensor 8 are against the perivaginal muscles, which is done thanks to the flange 5 that presses against the vulva, allowing its proper positioning.

In the case of the utilization of an open ring 50, the patient is placed on an examination bed or on her bed, said open ring is introduced into her vagina such that branches 51, 52 equipped with electro stimulation electrodes 56 are against the perivaginal (PV) muscles.

The housing is connected by a USB cable or by a Bluetooth system or by another system to a computer. The device is turned on and the physician proceeds with resetting the pressure sensor 8, either automatically after one minute of operation, or by using a virtual button situated on the screen of either the computer, or a PDA, or a mobile telephone allowing the housing to be controlled (off, on, reset, electro stimulation).

The electro stimulation system of the electronic card 15 is started, and the patient adjusts the intensity of the stimulation either by using a potentiometer placed on the housing or by using a virtual button situated on the screen of either the computer, or a PDA, or a mobile telephone.

The electro stimulation is continued until the end of the session, as with all muscle rehabilitation, since it improves the rehabilitation performance of the perivaginal (PV) muscles as well as the perivaginal vasodilation.

The patient must then verify that the voluntary contractions cause an elevation in pressure on pressure sensor 8 of probe 2, which proves that the right muscles are contracted, since only the perivaginal (PV) muscles should be contracted, without contraction of the abdominal muscles, which disturbs the measurements.

The patient then sees a mask of muscle contractions appear on the screen, that she is invited to follow, which indicates the optimal duration and height of the contraction that must be made.

This adjustable mask is established from the fatigue curve of the perivaginal (PV) muscles. It enables the rehabilitation to be optimized. In fact, insufficient work will be synonymous with the absence of improvement but, conversely, an overly intense rehabilitation will exhaust the perivaginal (PV) muscles.

Thus, an optimal intensity exists, calculated as a function of the fatigue curve. The fatigue of the perivaginal (PV) muscles is calculated by measuring the angle α formed on the curve.

For a given peak width, the smaller the angle, the greater the fatigue. Modifications in the pattern indicating the modalities of the contraction to be made will be calculated either in real time or during the session or between two sessions when this analysis will be carried out by the analysis and telecontrol center (ATC).

### Procedure for Treating Urinary Stress Incontinence Problems

Treatment is obtained by perineal rehabilitation carried out by means of measuring device 1 with electro stimulation of the vagina through electro stimulation means 7, 56 positioned on sensor 2, 50. This electro stimulation of the vagina enables the perivaginal (PV) muscles surrounding the vagina to be strengthened and the arterial circulation to be increased.

The rehabilitation may, for example, be done at the home of the patient, who has a probe 2 equipped with its electronic card 15 or an open ring 50 connected to the housing, not shown. The latter may be connected via Bluetooth to a computer or PDA or mobile telephone, as with the treatment of erection disorders.

The session is designed in the same manner as previously.

The patient is invited to place probe 2 or open ring 50 inside her vagina so that the electro stimulation plates 7, 56 and the pressure sensor are in contact with the perivaginal (PV) muscles. The patient turns the housing on and ensures that the connection is operating by verifying that a pressure variation (by pressing on the probe) results in a pressure elevation on the screen.

In addition, it must be understood that the previous description was only given by way of example and that it in no way limits the field of the invention from which one will not depart by replacing the details of embodiment described by any other equivalents.

1. A measuring method allowing vaginal and perivaginal physiological signals, such as pressures and pressure variations during contraction of the perivaginal (PV) muscles, but also perivaginal pulse, clitoral pulse, clitoral intracavernous pressure, vaginal temperature and any other relevant physiological signal to be measured, which comprises:
Placing a sensor (2, 50) comprising means for receiving the pressure and pressure variations during a contraction of the perivaginal (PV) muscles, and at least one transducer allowing the pressure signal issued from the pressure receiving means to be transformed into an electrical signal,
And connecting the sensor (2, 50) to an electronic card (15) or a housing comprising means for amplifying and recording the signal from the transducer, and means for analyzing the signal by a microcontroller allowing the state of perivaginal (PV) muscle fatigue to be measured and the variations in muscle strength of the perivaginal (PV) muscles to be measured.

2. The measuring method according to claim 1, further comprising measuring variations in intracavernous pressure (ICP) in order to quantify the variations in perivaginal tone and in arterial and venous circulation both day and night.

3. The measuring method according to claim 1, further comprising measuring the intracavernous pressure (ICP) when the pressure progressively reaches mean systolic pressure.

4. The measuring method according to claim 1, further comprising measuring the increase in arterial inflow in the clitoral corpus cavernosum and the reduction in venous outflow during the vascular phase, which is the first clitoral erection phase.

5. The measuring method according to claim 1, further comprising measuring variations in the contraction and rigidity of the perivaginal (PV) and erector (EM) muscles surrounding the clitoral corpus cavernosum during the muscular phase, which is the second clitoral erection phase.

6. The measuring method according to claim 1, further comprising continuously measuring the arterial inflow and the venous outflow by measuring the pulse pressure (PP) and the pulse volume (PV).

7. The measuring method according to claim 1, further comprising measuring the Young's modulus of the clitoral corpus cavernosum allowing the compliance of said clitoral corpus cavernosum to be evaluated.

8. The measuring method according to claim 1, further comprising measuring the leak from the intracavernous pressure (ICP) curve.

9. The measuring method according to claim 1, further comprising measuring the maximum contraction (Pmax), the pressure gradient (Delta P and mean delta), the surface under the curve of each contraction, the number of peaks and the width of each peak.

10. The measuring method according to claim 1, further comprising measuring perivaginal (PV) muscle fatigue by measuring the angle (α).

11. The measuring method according to claim 1, further comprising measuring the peaks of the perivaginal (PV) muscle contractions and the variations in pulse pressure.

12. A method for treating a patient with sexual dysfunctions, said patient having been diagnosed thanks to perivaginal and clitoral measurements taken both day and night, said measurements including measurements of perivaginal (PV) muscle strength and of arterial and venous variations, which comprises:
Placing a sensor (2, 50) comprising means for receiving the pressure and pressure variations during a contraction of the perivaginal (PV) muscles, means for electro stimulating (7, 56) the perivaginal (PV) muscles and at least one transducer allowing the pressure signal issued from the pressure receiving means to be transformed into an electrical signal,
And connecting the sensor (2, 50) to an electronic card (15) or to a housing comprising means for amplifying and recording the signal from the transducer, means for analyzing the signal by a microcontroller allowing the electro stimulation to be controlled as a function of the state of perivaginal (PV) muscle fatigue and the intensity and muscular rehabilitation to be controlled through sound, visual or tactile patterns indicating to the patient the intensity and duration of the muscle contraction to be produced and the duration of the rest period between two contractions.

13. The method for treating a patient with sexual dysfunctions according to claim 12, characterized in that the electro stimulation means are constituted of electrodes (7, 56) controlled by the patient in order to vary the intensity.

14. The method for treating a patient with sexual dysfunctions according to claim 12, characterized in that the diagnosis of organic dysfunction is established during the night by the measuring device (1).

15. The method for treating a patient with sexual dysfunctions according to claim 12, characterized in that the rehabilitation is specially indicated for patients presenting a muscular pathology having a normal vascular phase, but also for those having a mixed vascular pathology of arterial or venous and muscular origin.

16. The method for treating a patient with sexual dysfunctions according to claim 12, characterized in that the patient is placed on an examination bed or on her bed, the probe (2) is introduced into her vagina such that the electro stimulation plates (7) and the pressure sensor (8) are against the perivaginal (PV) muscles, which is achieved thanks to the flange (5) that presses on the vulva, allowing it to be positioned properly.

17. The method for treating a patient with sexual dysfunctions according to claim 12, characterized in that the patient is placed on an examination bed or on her bed, the open ring (50) is introduced into her vagina such that the branches (51, 52) equipped with electro stimulation electrodes (56) are against the perivaginal (PV) muscles.

18. The method for treating a patient with sexual dysfunctions according to claim 12, characterized in that the electro stimulation system of the electronic card (15) or the housing is started, and the patient adjusts the intensity of the stimulation either by using a potentiometer placed on the housing or by using a virtual button situated on the screen of either the computer, a PDA, or a mobile telephone.

19. The method for treating a patient with sexual dysfunctions according to claim 12, characterized in that the electro stimulation is continued until the end of the session, as with any muscular rehabilitation, since it improves the performance of the perivaginal (PV) muscle rehabilitation as well as the perivaginal vasodilation.

20. The method for treating a patient with sexual dysfunctions according to claim 12, characterized in that the patient verifies that the voluntary contractions cause an elevation in pressure on pressure sensor (2, 50), which proves that the right muscles are contracted, since only the perivaginal (PV) muscles should be contracted, without contraction of the abdominal muscles, which disturbs the measurements.

21. The method for treating a patient with sexual dysfunctions according to claim 12, characterized in that the patient then sees a mask of muscle contractions on the screen that she is invited to follow, indicating the optimal duration and height of the contraction that must be made.

22. The method for treating a patient with sexual dysfunctions according to claim 12, characterized in that the muscle contraction mask is adjustable and established from the fatigue curve of the perivaginal (PV) muscles.

23. The method for treating a patient with sexual dysfunctions according to claim 12, characterized in that modifications in the mask indicating the modalities of the contraction to be performed will be calculated either in real time by the microcontroller during the session or between two sessions when this analysis will be done by the analysis and telecontrol center (ATC).

24. A method for treating a patient with problems with urinary stress incontinence according to claim 12, characterized in that perineal rehabilitation is obtained by means of the sensor (2, 50) with electro stimulation of the vagina through electro stimulation means (7, 56) positioned on the sensor (2, 50), electro stimulation of the vagina allowing the perivaginal (PV) muscles surrounding the vagina to be strengthened and the arterial circulation to be increased.

25. A measuring device for implementing the method according to claim 1, characterized in that the sensor is constituted of a probe (2) comprising a cylindrical longitudinal body (4) equipped with a flange (5) forming a stop, a pressure sensor (8) placed according to a direction longitudinal to the surface of the cylindrical body (4), an electronic card (15) disposed inside the cylindrical body (4) and to which the pressure sensor (8) is connected, and communication means longitudinally traversing the probe (2) that are constituted of at least one longitudinal channel (16) provided on the periphery of the cylindrical body (4) and traversing the flange (5) to enable the inner part of the vagina to be connected to the outer in order to balance the intravaginal and extravaginal pressures.

26. The measuring device for implementing the method according to claim 1, characterized in that the sensor is constituted of a probe (2) comprising a cylindrical longitudinal body (4) equipped with a flange (5) forming a stop, a pressure sensor (8) placed according to a direction longitudinal to the surface of the cylindrical body (4), an electronic card (15) disposed inside the cylindrical body (4) and to which the pressure sensor (8) is connected, and communication means longitudinally traversing the probe (2) that are constituted of a channel (17) provided inside the cylindrical body (4) and longitudinally traversing the latter and the flange (5) to enable the inner part of the vagina to be connected to the outer in order to balance the intravaginal and extravaginal pressures.

27. The measuring device for implementing the method according to claim 1, characterized in that the sensor is constituted of a probe (2) comprising a longitudinal cylindrical body (4) equipped with a flange (5) forming a stop, a pressure sensor (8) placed according to a direction longitudinal to the surface of the cylindrical body (4), an electronic card (15) disposed inside the cylindrical body (4) and to which the pressure sensor (8) is connected, and communication means longitudinally traversing the probe (2) that are constituted of, first, at least one longitudinal channel (16) provided on the periphery of the cylindrical body (4) and traversing the flange (5) and, second, by a channel (17) provided inside the cylindrical body (4) and longitudinally traversing the latter and the flange (5), said communication means allowing the inner part of the vagina to be connected to the outer in order to balance the intravaginal and extravaginal pressures.

28. A measuring device for implementing the method according to claim 25, characterized in that the probe (2) comprises, at the surface of the cylindrical body (4), electro stimulation plates (7) connected to the electronic card (15).

29. The measuring device according to claim 28, characterized in that the electro stimulation plates (7) are positioned parallel to the longitudinal axis of said cylindrical body (4).

30. The measuring device according to claim 28, characterized in that the electro stimulation plates (7) present a circular or spherical profile.

31. The measuring device according to claim 25, characterized in that the pressure sensor (8) is constituted of a metallic plate (9) that is fixed in a groove (10) provided in the cylindrical body (4), said groove (10) being filled with a composite elastic material (12) allowing the movements or elastic deformations of this plate (9) to be absorbed, while the inner face of the metallic plate (9) is integral with a strain gauge (11) or transducer allowing the pressure signal issued from the metallic plate (9) to be transformed into an electrical signal.

32. The measuring device according to claim 25, characterized in that the pressure sensor (8) is constituted of a piezoelectric film (13) or transducer fixed at the level of the groove (10) filled with a composite elastic material (12).

33. The measuring device according to claim 25, characterized in that the probe (2) comprises, inside the cylindrical body, (4) a cutout (14) permitting an electronic card 15 to be placed and held.

34. The measuring device according to claim 25, characterized in that the probe (2) comprises all the sensors necessary for the vaginal physiological measurements connected with the electronic card (15).

35. The measuring device according to claim 25, characterized in that the electronic card (15) may be connected to a housing containing an amplifier, an analog to digital converter and a microprocessor.

36. A measuring device for implementing the measuring method according to claim 1, characterized in that the sensor is constituted of:
An open ring (50) comprising two branches (51, 52) in an arc of circumference interconnected by a flexible element (53),
And a strain gauge (55) integral with the flexible element (53) and connected to a housing or to an electronic card.

37. The measuring device according to claim 36, characterized in that the strain gauge (55) is disposed on the inner face of the flexible element (53) such that the latter is found inside the open ring (50) to prevent any risk of injuring the vagina of the patient.

38. The measuring device according to claim 37, characterized in that the branches (51, 52) are plate-shaped flat branches made in a material with high elastic deformation strength, or a material that is said to be not very deformable.

39. The measuring device according to claim 37, characterized in that the flexible element (53) connecting the branches (51, 52) is constituted of a thin plate of metal presenting significant elastic deformation characteristics.

40. The measuring device according to claim 36, characterized in that the open ring (50) comprises, on the outer face of branches (51, 52), electro stimulation means that are constituted of electrodes (56) connected to a housing containing an amplifier, an analog to digital converter, an electronic card, a microprocessor and a microcontroller managing the electro stimulation.
